(19) United States
(12) Patent Application Publication
(10) Pub. No.: US 2005/0065596 A1
(43) Pub. Date: Mar. 24, 2005

(54) Stents capable of controllably releasing histone deacetylase inhibitors

(76) Inventors: Xufan Tseng, New York, NY (US); Shuyun Xu, Anhui Province (CN)

Correspondence Address: PATTON BOGGS LLP, 8484 WESTPARK DRIVE, SUITE 900, MCLEAN, VA, 22102, US

(21) Appl. No.: 10/489,859

(22) PCT Filed: Jul. 18, 2003
(86) PCT No.: PCT/US03/22449

Related U.S. Application Data
(60) Provisional application No. 60/397,780, filed on Jul. 24, 2002.
(60) Provisional application No. 60/402,086, filed on Aug. 9, 2002.

(51) Int. Cl. A61F 2/06

(52) U.S. Cl. 623/1.42

(57) ABSTRACT

A stent device is provided. The stent device includes a stent body and one or more HDAC inhibitor depot(s) provided on or in the stent body, the depot(s) capable of controllably releasing HDAC inhibitor(s). Methods of using the stents in treating and/or preventing restenosis are provided. A delivery system including the stent device and a methods of using the delivery system in treating and/or preventing restenosis are also provided. Kits comprising stents are provided.

### FIELD AND BACKGROUND OF THE INVENTION

The present invention relates to a stent device effective in preventing restenosis and, more particularly, to a stent device that is designed to controllably release HDAC inhibitors, to thereby prevent and/or treat restenosis.

Histones are a family of small, positively charged (at physiological pH) proteins which are rich in basic amino acids and are generally highly conserved across eukaryotic species. There are four classes of histones, termed H2A, H2B, H3, and H4, which associate to form a disk-shaped octomeric protein core.

In eukaryotic cells, genomic DNA associates with histones, as well as with other proteins, to form a compact complex called chromatin. The DNA winds around the protein core of the nucleosome, such that the basic, positively charged, amino acids of the histones interact with the negatively charged phosphate groups of the DNA.

Approximately 146 base pairs of DNA wrap around a histone core to make up a nucleosome particle, which constitutes the repeating structural motif of chromatin.

A small fraction of histones, more specifically, the amino side chains thereof, are enzymatically modified by post-translational addition of methyl, acetyl, or phosphate groups, which either neutralizes the positive charge of the side chain, or converts it to a negative charge.

For example, lysine and arginine residues may be methylated, lysine residues may be acetylated, and serine residues may be phosphorylated. For lysine, the —(CH₂)₄—NH₂ side-chain may be acetylated, for example, by an acetyltransferase enzyme, to give the amide —(CH₂)₄—NHC(═O)CH₃. When enzymatically added to the amino termini of histones, these methyl, acetyl, and phosphate groups extend from the nucleosomal core, thereby affecting the chromatin structure and hence affecting gene expression (see, for example, Spencer and Davie, 1999).

More specifically, these enzymatic modifications affect the chromatin structure by altering the electrostatic charge and thereby specifying a pattern that is recognized by chromatin associated regulatory proteins. The binding of these regulatory proteins, and optionally of other proteins, to chromatin affects is the loosening level of the chromatin structure.

It has been found that acetylation and deacetylation of histones is associated with transcriptional events leading to cell proliferation, apoptosis and/or differentiation. (For recent reviews of histone acetylation and deacetylation, see, for example, Kouzarides, 1999 and Pazin et al., 1997).

Certain enzymes, specifically acetylases (e.g., histone acetyltransferase, HAT) and deacetylases (e.g., histone deacetylase, HDAC), which regulate the acetylation state of histones, have been identified in many organisms and have been implicated in the regulation of gene expression, confirming the link between acetylation and transcription (see, for example, Davie, 1998).

In normal cells, histone deacetylase (HDAC) and histone acetyltransferase (HAT) together control the level of acetylation of histones to maintain an acetylation/deacetylation balance.

Csordas (1990) teaches that histones are subject to post translational acetylation of the ε-amino groups in N-terminal lysine residues, a reaction that is catalyzed by histone acetyl transferase (HAT). Acetylation neutralizes the positive charge of the lysine side chain, and is thought to impact chromatin structure. Taunton et al. (1996) teach that access of transcription factors to chromatin templates is enhanced by histone hyperacetylation. Taunton et al. further teach that an enrichment in under-acetylated histone H4 has been found in transcriptionally silent regions of the genome.

Histone acetylation is a reversible modification, with deacetylation being catalyzed by a family of enzymes termed histone deacetylases (HDACs). Marks et al. (2001a) teach that three classes of HDACs have been identified so far. The first class is represented by yeast Rpd3-like proteins, and the second class is represented by yeast Hda1-like proteins (Grozinger et al. 1999). Human HDAC1, HDAC2, and HDAC3 proteins are members of the first class of HDACs, while human HDAC4, HDAC5 and HDAC6, are members of the second class of HDACs (Grozinger et al. 1999). Van den Wyngaert (2000) discloses HDAC8, a new member of the first class of HDACs. Kao et al. (2000) disclose HDAC7, a new member of the second class of HDACs. Marks et al. (2001a) also teach another member of the second HDAC class, named HDAC9. The third HDAC class is represented by homologues to yeast and mouse Sir2. An additional class of HDACs, which includes Hos1, Hos2 and Hos3, has also been identified in yeast. However, homologues of these new HDACs have not been found in mammalian tissues.

The important role of histone acetylation and deacetylation in cell apoptosis, proliferation and differentiation is demonstrated, for example, in Marks et al. (2001a-b), Yoshida et al. (2001), Jung (2001), Wang et al. (2001), Marks et al. (2000), and Weidle et al. (2000). These studies demonstrate direct correlation between histone acetylation and cell proliferation. In particular, these studies teach that deregulation of histone acetylation causes uncontrolled cell proliferation and thereby promotes pathological developments such as cancer and keloid formation (a fibroproliferative disorder following a dermal injury).

In view of the important role of HDACs in cell proliferation and differentiation, studies of HDAC inhibitors have been conducted. Inhibition of HDACs results in the accumulation of hyperacetylated histones, which results in a variety of cellular responses. Upon acetylation, highly charged histone is neutralized, loses its contact with DNA, and generates an open DNA conformation that is more accessible by transcription factors, regulatory complexes, and RNA polymerase. HDAC inhibitors lead to histone hyperacetylation, and hence relax the structure of chromatin associated with a set of programmed genes, regulate specific gene expression, and arrest abnormal cell growth. It has been found that HDAC inhibitors induce growth arrest, differentiation, and/or apoptotic cell death in a variety of transformed cells (e.g., cancer cells).

Richon et al. (1998) disclose that HDAC activity is inhibited by s trichostatin A (TSA), a natural product isolated from Streptomyces hygroscopicus, and by a synthetic compound, suberoyl anilide hydroxamic acid (SAHA). Yoshida and Beppu (1988) teach that TSA causes arrest of rat fibroblasts at the G₁ and G₂ phases of the cell cycle, implicating HDACs in cell cycle regulation. Yoshida et al. (1990) teach that nanomolar concentrations of TSA cause a marked accumulation of highly acetylated histones in vivo and strongly inhibit purified HDAC in vitro. Finnin et al. (1999) teach that TSA and SAHA inhibit cell growth, induce terminal differentiation, and prevent the formation of tumors in mice. Marks et al. (2000, 2001) and Kelly et al. (2001) teach that hydroxamic acid-based histone deacetylase inhibitors, such as SAHA and TSA, limit tumor cell growth in animals with little or no toxicity. Vigushin et al. (2001) teach that TSA has potent dose-dependent anti-tumor activity against breast cancer, both in vitro and in vivo. Koyama et al. (2000) and Takahashi et al. (1996) teach that TSA acts as an immunosuppressive agent by inhibiting IL-2 gene expression. Ailenberg et al. (2002) and Liu et al. (2003) teach that TSA exhibits antimigratory activity by inhibiting metalloproteinases (MMPs). Trichostatin A has also been reported to be useful in the treatment of fibrosis, e.g., liver fibrosis and liver cirrhosis (EP 0827743). Okabe et al. (1995) and Kimura et al. (1994) teach that TSA inhibits PDGF as well as bFGF in primary smooth muscle cells (SMCs) of rat aorta. Recently, it has been reported (Skaletz-Rosowski et al., 2000) that TSA, as well as a new synthetic HDAC inhibitor named M232, inhibit the proliferation of coronary SMCs.

Finnin et al. (1999) disclose that each of the HDAC inhibitors TSA and SAHA binds HDAC by inserting its aliphatic chain into the catalytic pocket, thereby making multiple contacts to the tube-like hydrophobic portion of the pocket (see FIG. 15 for the chemical structure of TSA). The hydroxamic acid group reaches the polar bottom of the pocket, coordinates the zinc and also contacts active-site residues. The chelation of the zinc is thought to be the main mechanism of inhibition. The aromatic ring serves as a cap necessary for packing the inhibitor at the rim of the tube-like active-site pocket. The length of the aliphatic chain appears to be optimal for spanning the length of the pocket and allowing contacts both at the bottom and at the entrance of the pocket.

TSA and SAHA are both hydroxamic acids. As is well known and widely used in the art, the phrase “hydroxamic acid” describes a compound having a “—C(═O)—NHOH” group.

Other compounds that belong to the class of hydroxamic acids, such as oxamflatin (Kim et al., 1999), CBHA (m-carboxycinnamic acid bishydroxamide, Richon et al., 1998), CHAP1 (cyclic hydroxamic acid-containing peptide 1), CHAP31, SBHA (suberic bishydroxamate), pyroxamide, scriptaid and other hydroxamic acids disclosed, for example, in WO 02/22577, WO 01/70675 and WO 01/38322, have been reported to inhibit HDAC.

Although a wide variety of the presently known HDAC inhibitors are classified as hydroxamic acids, it has been reported that compounds of other structural classes also inhibit HDAC. The main structural classes to which HDAC inhibitors are typically related, in addition to the hydroxamic acids class, include: short-chain fatty acids such as butyrate, phenyl butyrate and other butyrate derivatives (Lea and Tulsyan, 1995); cyclic tetrapeptides containing a 2-amino-8-oxo-9,10-epoxy-decanoyl (AOE) moiety, such as trapoxin, trapoxin A, chlamydocin and HC toxin; cyclic tetrapeptides not containing the AOE moiety, such as FR901228 (Nakajima et al., 1998), WF 27082 (WO 00/21979) and apicidin; and benzamide derivatives, such as MS-27-275 (Saito et al. (1999); Suzuki et al. (1999)).

The natural products trapoxin and HC-toxin, which represent a family of HDAC inhibitors that is chemically distinct from hydroxamic acids, contain groups that may be analogous to the cap, aliphatic chain and zinc-binding groups of TSA. The epoxy group may function as hydroxamic acid by crosslinking to an active site nucleophile. The ketone group may interact with polar residues, and possibly the zinc. The cyclic tetrapeptide contains hydrophobic groups and may serve as a cap. The aliphatic chain probably binds in the hydrophobic tube-like portion of the pocket. (See Finnin et al. (1999)).

Other compounds reported as HDAC inhibitors include diallyl sulfide and related molecules (Lea et al., 1999), depudecin (Kwon et al., 1998), psammaplin A, valproic acid and derivatives thereof (EP 1170008), carbamic acid derivatives (WO 02/30879, WO 02/26696 and WO 02/26703) and derivatives of oxyalkylene esters and oxyallcylene phosphates (see, e.g, U.S. Pat. Nos. 6,043,389, 6,030961 and 6,239,176). All the above references teaching various HDAC inhibitors are incorporated by reference as if fully set forth herein.

In vitro, some of these compounds are reported to inhibit the growth of fibroblast cells by causing cell cycle arrest in the G₁ and G₂ phases, and can therefore lead to the terminal differentiation and loss of transforming potential of a variety of transformed cell lines (see, e.g., Richon et al., 1996; Kim et al., 1999; Yoshida et al., 1995; Yoshida and Beppu, 1988). In vivo, phenylbutyrate is reported to be effective in the treatment of acute promyelocytic leukemia in conjunction with retinoic acid (see, e.g., Warrell et al., 1998). Butyric acid and its derivatives, including sodium phenylbutyrate, have been reported to induce apoptosis, in vitro, in human colon carcinoma, leukemia and retinoblastoma cell lines. SAHA is reported to be effective in preventing the formation of mammary tumors in rats, and lung tumors in mice (see, e.g., Desai et al., 1999).

Although these findings suggest that inhibition of HDAC activity represents a novel approach for intervening in cell cycle regulation and that HDAC inhibitors have great therapeutic potential in the treatment of cell proliferative diseases or conditions, it has been found that most of the presently known HDAC inhibitors are not useful pharmacological agents, mainly due to the in vivo instability and short half-life of the compounds.

Hence, it is highly desirable to develop systems that are directed toward increasing the pharmacological efficiency of HDAC inhibitors.

Restenosis is the closure of a peripheral or coronary artery following trauma to the artery caused by efforts to open an occluded portion of the artery.

Restenosis is typically caused by trauma inflicted during angioplasty, effected by, for example, balloon dilation, atherectomy, or laser ablation treatment of the artery. Although angioplasty procedures are routinely used in treatment of occluded arteries, appearance of restenosis following angioplasty treatment remains a significant problem. For these angioplasty procedures, restenosis occurs at a rate of about 30% to about 60% depending upon the vessel location, lesion length, and a number of other variables. For example, studies have shown that within three to six months after a balloon angioplasty, between 25% and 45% of patients experience a re-narrowing of the artery (restenosis). One aspect of restenosis may be simply mechanical; e.g., caused by the elastic rebound of the arterial wall and/or by dissections in the vessel wall caused by the angioplasty procedure. These mechanical problems have been successfully addressed by the use of stents to tack-up dissections and prevent elastic rebound of the vessel, thereby reducing the level of restenosis for many patients.

Stents are typically metallic or polymeric devices that are permanently implanted in an expanded form in coronary and peripheral vessels. A stent is typically inserted by a catheter into a vascular lumen and expanded into contact with the arterial wall, thereby providing internal support for the lumen. Examples of stents are disclosed in U.S. Pat. Nos. 4,733,665, 4,800,882 and 4,886,062.

Although stents are routinely used in clinical procedures, and stents have proven useful in preventing and treating restenosis, clinical data show that stents are not capable of completely preventing in all patients in-stent restenosis (ISR) or restenosis caused by intimal hyperplasia.

In-stent restenosis is the reoccurrence of the narrowing of an artery following stent implantation. It is estimated that about 20% of patients treated with coronary stents suffer from in-stent restenosis. Intimal hyperplasia results from migration and proliferation of medial SMCs following arterial wall injury caused by angioplasty. Such proliferation leads to occlusion of the artery.

Many pharmacological attempts have been made to reduce the amount of restenosis caused by intimal hyperplasia. Many of these attempts have dealt with the systemic delivery of drugs via oral or intravascular introduction. However, success with the systemic approach has been limited.

Systemic delivery of drugs is inherently limited since it is difficult to achieve constant drug delivery to the inflicted region and since systemically administered drugs often cycle through concentration peaks and valleys, resulting in time periods of toxicity and ineffectiveness. Therefore, in order to be effective, anti-restenosis drugs should be delivered in a localized manner.

One approach for localized drug delivery utilizes stents as delivery vehicles. For example, stents seeded with transfected endothelial cells expressing bacterial beta-galactosidase or human tissue-type plasminogen activator were utilized as therapeutic protein delivery vehicles (Dichek et al., 1989).

U.S. Pat. No. 5,679,400, International Patent Application WO 91/12779, entitled “Intraluminal Drug Eluting Prosthesis,” and International Patent Application WO 90/13332, entitled “Stent With Sustained Drug Delivery” disclose stent devices capable of delivering antiplatelet agents, anticoagulant agents, antimigratory agents, antimetabolic agents, and other anti-restenosis drugs.

U.S. Pat. Nos. 6,273,913, 6,383,215, 6,258,121, 6,231,600, 5,837,008, 5,824,048, 5,679,400 and 5,609,629 teach stents coated with various pharmaceutical agents such as Rapamycin, 17-beta-estradiol, Taxol and Dexamethasone.

Although prior art references disclose numerous stents configurations coated with one or more distinct anti-restenosis agents, none disclose or suggest stents capable of controllably releasing HDAC inhibitors for the purpose of preventing and/or treating restenosis.

### SUMMARY OF THE INVENTION

Because HDAC inhibitors have anti-proliferative activity and have shown great therapeutic potential in the treatment of cell proliferative diseases or conditions, the controllable release thereof from a stent would be highly advantageous in the treatment and/or prevention of restenosis.

According to the present invention, there is provided a novel stent device, which is highly beneficial in prevention and treatment of restenosis. The stent device of the present invention is designed to controllably release one or more HDAC inhibitors, which are efficient inhibitors of SMC proliferation.

Hence, according to one aspect of the present invention there is provided a stent device comprising a stent body and one or more HDAC inhibitor depot(s) on or in the stent body, the one or more HDAC inhibitor depot(s) being capable of controllably releasing one or more HDAC inhibitor(s).

According to another aspect of the present invention there is provided a catheter device comprising a catheter and a stent device of the present invention. In such embodiments, the stent device is mounted on or in the catheter.

The present invention also provides kits comprising a stent or stents according to the present invention and a delivery system suitable for positioning the stent(s) within a lumen or lumens. The delivery system of a kit according to the present invention may include a delivery catheter. In kits according to the present invention, the stent(s) may be mounted in or on the delivery system. Alternatively, the stent(s) may be separate from the delivery system and may be mounted therein or thereon prior to use.

The stent device of the present invention, optionally as a part of the catheter device described herein, is highly efficient in preventing restenosis and/or in treating restenosis. Hence, according to yet another aspect of the present invention, there is provided method of preventing and/or treating restenosis in a lumen of a subject in need thereof The method comprises positioning in the lumen the stent device of the present invention. The lumen may be, for example, an arterial lumen.

According to another embodiment of the invention, at least one of the one or more HDAC inhibitor depots further comprises one or more additional pharmaceutical agent(s) and is capable of controllably releasing the additional pharmaceutical agent(s).

According to still other embodiments of the invention, the body of a stent device according to the invention further comprises one or more additional pharmaceutical agent depot(s) on or in the stent body, wherein the one or more additional pharmaceutical agent depot(s) is capable of controllably releasing at least one additional pharmaceutical agent(s).

According to some embodiments, the one or more additional pharmaceutical agent(s) is selected from the group consisting of an anticoagulant agent, an antiproliferative agent, an antimigratory agent, an antimetabolic agent, an anti-inflammatory agent, and an immunosuppressive agent.

According to some embodiments of the invention, at least one of one or more the HDAC inhibitor depot(s) comprises one or more biocompatible polymer(s) loaded with one or more HDAC inhibitor(s); and at least one of the one or more pharmaceutical agent depot(s) comprises one or more biocompatible polymer(s) loaded with the one or more pharmaceutical agent(s).

According to other embodiments, at least one of the one or more biocompatible polymer(s) comprises a biostable polymer and/or a biodegradable polymer.

According to some embodiments, at least one of the one or more HDAC inhibitor(s) is a hydroxamic acid, such as, but not limited to, TSA, SAHA, oxamflatin, CBHA, CHAP1, CHAP31, SBHA, pyroxamide and scriptaid.

According to still other embodiments at least one of the one or more HDAC inhibitor(s) is selected from the group consisting of a short-chain fatty acid, a cyclic tetrapeptide, a benzamide derivative, a valproic acid derivative, a carbamic acid derivative, an allyl sulfide-containing compound, an oxyalkylene ester derivative and an oxyalkylene phosphate derivative.

The cyclic tetrapeptide can be, for example, trapoxin, trapoxin A, chlamydocin or HC toxin. Alternatively, the cyclic tetrapeptide can be FR901228, WF 27082, or apicidin.

According to yet other embodiments, the body of a stent according to the present invention comprises metal, plastic, or both.

According to still other embodiments, the body of a stent according to the present invention is self-expansible. In some embodiments, a self-expansible stent body according to the present invention is mounted in or on a catheter.

According to still other embodiments, the body of a stent according to the present invention is forcedly expansible. In some embodiments, a forcedly expansible stent body according to the present invention is mounted in or on a catheter.

According to some embodiments, at least one of the one or more HDAC inhibitor depot(s) is on an external surface of the stent body.

According to some embodiments, at least one of the one or more HDAC inhibitor depot(s) is on an internal surface of the stent body.

According to some embodiments, at least one of the one or more HDAC inhibitor depot(s) is within a wall of the stent body. As a non-limiting example, HDAC depot(s) may be present within a stent wall comprising a biodegradable polymer.

According to some embodiments, at least one of the one or more additional pharmaceutical agent depot(s) is on an external surface of the stent body.

According to some embodiments, at least one of the one or more additional pharmaceutical agent depot(s) is on an internal surface of the stent body.

According to some embodiments, at least one of the one or more additional pharmaceutical agent depot(s) is within a wall of the stent body. As a non-limiting example, HDAC depot(s) may be present within a stent wall comprising a biodegradable polymer.

The present invention successfully addresses the shortcomings of the presently known configurations by providing a stent device capable of controllably releasing HDAC inhibitors and hence is capable of efficiently inhibiting SMC proliferation and thus preventing and/or treating restenosis.

Unless otherwise defined, all technical and scientific terms used herein have the same meaning as commonly understood by one of ordinary skill in the art to which this invention belongs. Although methods and materials similar or equivalent to those described herein, or otherwise known in the art to be suitable, can be used in the practice or testing of the present invention, exemplary suitable methods and materials are described below. The materials, methods, and examples set forth herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Equivalent embodiments of the invention will be apparent to one of skill in the art upon reading the present inventive disclosure; and such equivalent embodiments are encompassed by the claims appended hereto.

In the figures:

FIGS. 1a-b are schematic perspective views of an exemplary self-expansible stent;

FIG. 2 is a schematic cross-sectional view of an exemplary catheter having a self-expansible stent mounted therein;

FIG. 3 is a schematic cross-sectional view of an exemplary forcedly expansible stent configuration;

FIG. 4 is a schematic cross-sectional view of an exemplary catheter having a forcedly expansible stent mounted thereon;

FIGS. 5a-c are schematic cross-sectional views of a stent device having a HDAC on the stent body (FIGS. 5a and 5c) or within the stent body (FIG. 5b);

FIG. 6 diagrammatically presents the induction by HDAC inhibitors of growth arrest and/or apoptosis in uncontrolled cell growth;

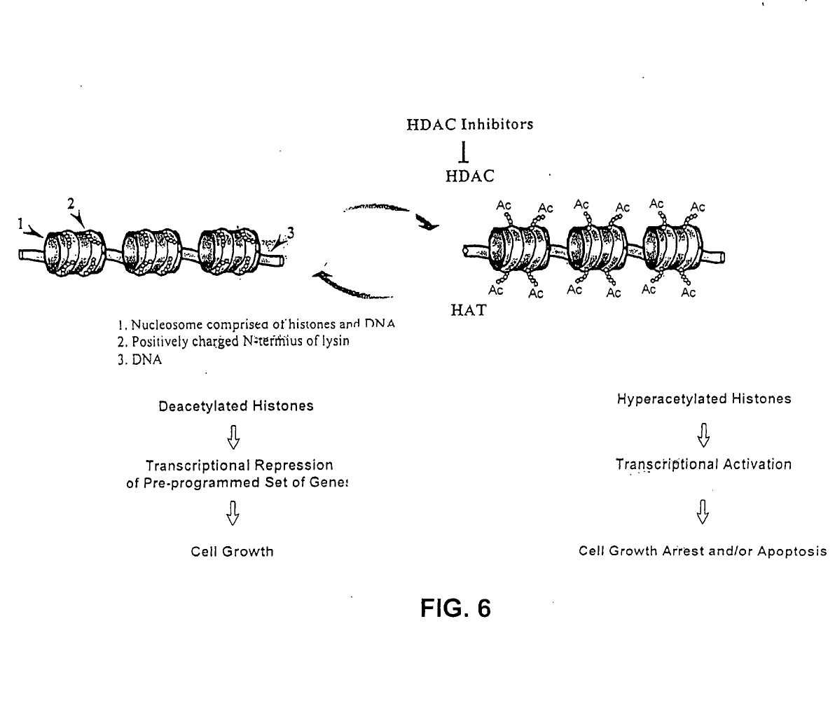

FIG. 7 presents the chemical structures of exemplary HDAC inhibitors classified as hydroxamic acids;

FIG. 8 presents the chemical structures of exemplary HDAC inhibitors i classified as short-chain fatty acids;

FIG. 9 presents the chemical structures of exemplary HDAC inhibitors classified as cyclic tetrapeptides containing an AOE moiety;

FIG. 10 presents the chemical structures of exemplary HDAC inhibitors classified as cyclic tetrapeptides not containing an AOE moiety;

FIG. 11 presents the chemical structures of additional exemplary HDAC inhibitors;

FIG. 12 presents the morphological analysis performed after 1-day incubation of human aortic SMCs with TSA, RPM and Paclitaxel, compared with untreated cells;

FIG. 13 presents bar graphs demonstrating the inhibitory effect of TSA and RPM on the growth of human aortic SMCs; and

FIG. 14 presents comparative growth curves of human aortic SMCs treated with 50 nM TSA and untreated SMCs.

FIG. 15 presents the chemical structure of Trichostatin A (TSA), an exemplary HDAC inhibitor.

### DETAILED DESCRIPTION OF THE INVENTION

The present invention relates to a stent device that can be used to prevent and/or treat restenosis. In certain embodiments, the invention also includes a delivery system for the stent device. Specifically, the present invention provides stent devices and methods of preventing and/or treating restenosis using the same; in certain embodiments, the present invention also provides a delivery system for the stent device and methods of preventing and/or treating restenosis using the same. Stent devices according to the present invention are designed to controllably release HDAC inhibitor(s), which are capable of efficiently inhibiting SMC proliferation thereby preventing restenosis.

The principles and operation of a stent and a catheter device according to the present invention may be better understood with reference to the drawings and accompanying descriptions.

The invention is not limited in its application to the details of construction and the arrangement of the components set forth in the following description or illustrated in the drawings. The invention is capable of other embodiments or of being practiced or carried out in various ways, as would be apparent to one skilled in the art upon reading the present inventive disclosure. Also, it is to be understood that the phraseology and terminology employed herein is for the purpose of description and should not be regarded as limiting.

As is discussed herein in the Background section, restenosis involves a natural healing reaction to the injury of an arterial wall. In many cases, this healing process results in migration and proliferation of medial SMCs, which finally cause re-occlusion of the artery, known as restenosis.

There are several recognizable routes for biochemical prevention and treatment of restenosis. These routes include, for example, (1) reducing the adhesion and aggregation of platelets at the arterial injury site; (2) blocking the expression and/or activity of growth factors and/or their receptors; (3) and interfering with the activation of signaling pathways in responsive cells.

Reduction of the adhesion and aggregation of platelets is directly related to reduction of the formation of thrombus, a major problem associated with all types of angioplasty procedures. The second and third routes address the downregulation of cytokines, growth factors, and other endogenous target proteins, which are produced from sources other than platelets, to reduce or eliminate SMC proliferation and/or migration and/or inflammation at the artery injury site.

As is discussed herein and further exemplified in the Examples section that follows, it has been found that HDAC inhibitors act as strong and potent inhibitors of SMC proliferation. The present invention discloses a stent provided with a HDAC inhibitor depot, which is designed to controllably release the HDAC inhibitor, thus providing a novel and effective approach to the prevention and treatment restenosis.

As used herein, the term “preventing” includes stopping or reducing the occurrence or severity of a disease or condition or the symptoms of the disease or condition.

As used herein, the term “treating” includes substantially reducing the severity of a disease or condition or the symptoms of the disease or condition, or substantially reducing the appearance of a disease or condition or the symptoms of the disease or condition. The term “treating” includes substantially completely abolishing a disease or condition or the symptoms of the disease or condition. The term “treating” also encompasses preventing, stopping, or reducing the occurrence or severity of a disease or condition or the symptoms of the disease or condition.

The phrases “controllably release”, “controllable release,” and “controllably releasing” are used herein to describe a release of an agent (e.g., HDAC inhibitor) at a predetermined rate and duration under selected conditions. Slow release is one form of controllable release.

According to one aspect of the present invention, there is provided a stent device. The stent device of the present invention comprises a stent body and a HDAC inhibitor depot for controllably releasing a HDAC inhibitor.

The stent body of the present invention can be any stent or prosthesis that is usable in transluminal applications, or, in other words, any stent or prosthesis which may be inserted and positioned where desired in a vessel lumen.

The stent device of the present invention can be fabricated from a metal, such as stainless steel, tantalum, titanium alloy, cobalt alloy, silicones or a polymer such as, thermoplastic elastomers including, but not limited to, polyolefin elastomers and polyamide elastomers or any combinations thereof

Stents fabricated from a metal generally have better mechanical properties than polymeric stents, and thus may be preferred in certain embodiments. Metallic stents typically provide a large amount of radial strength to resist inwardly directed circumferential pressure in blood vessels. In order for a polymer material to provide comparable strength characteristics, a much thicker-walled structure or heavier, denser filament weave is required. This, in turn, reduces the cross-sectional area available for flow through the stent and/or reduces the relative amount of open space available in the structure. In addition, it may be more difficult to load and deliver polymeric stents using catheter devices. Accordingly, in certain embodiments of the invention, metal stents are preferred.

Stent devices are generally designed as permanent implants, which may become incorporated in the vascular or other tissue they contact at implantation.

Typically, stent devices are applied and/or positioned in a desired location, utilizing a vascular catheter or any other similar transluminal device.

Hence, according to another aspect of the present invention, there is provided a delivery system, which includes the stent device described herein and a delivery catheter. The delivery catheter and stent device are configured to be detachably attached to one another.

Kits comprising a stent or stents according to the present invention and a delivery system suitable for positioning the stent(s) within a lumen or lumens are also provided. The delivery system of a kit according to the present invention may include a delivery catheter.

The delivery catheter serves for positioning the stent device within the lumen area of interest. As such, when attached to the delivery catheter, the stent device preferably assumes a contracted configuration which facilitates delivery. Upon positioning, the delivery catheter can be used to forcibly expand the stent device into an expanded position which anchors the stent device within the lumen area. Following anchoring, the delivery catheter is released from the stent device and removed. Alternatively, the stent device can be configured to self expand following release from the delivery catheter and to thereby self-anchor within the lumen area.

Hence, the stent body, according to the present invention, can be either self-expansible or forcedly expansible.

Self-expansible stents are typically flexible tubular bodies that can be narrowed and elongated upon axial tension and revert to stable dimensions upon relaxation. Examples of self-expansible stents suitable for use according to the present invention are those that are made of Nitinol or any other self-expansible metals or metals having thermal memory. Hence, stent devices that have a self-expansible stent body are typically mounted in a catheter, in their elongated configuration, and expand upon release from the catheter, typically to a predetermined diameter.

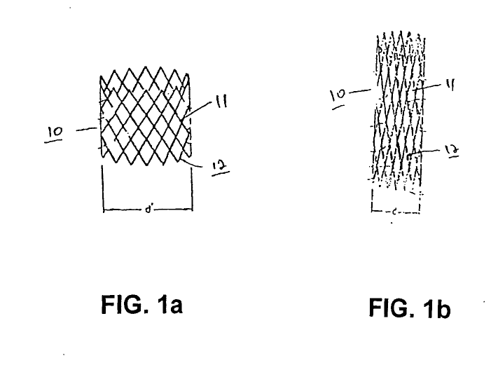

As an example, FIGS. 1a-b illustrate a self-expansible stent, which is referred to herein as stent 10. Stent 10 has a flexible tubular stent body 11 formed of several individual flexible thread elements 12, each of which extends in a helix configuration with the centerline of the body serving as a common axis. The elements are wound in a common direction, but are displaced axially relative to each other and meet, under crossing a like number of elements also so axially displaced, but having the opposite direction of winding. This configuration provides a resilient braided tubular structure, which assumes stable dimensions upon relaxation, as is shown in FIG. 1a. Axial tension produces elongation and corresponding diameter contraction that allows the stent to be mounted in a catheter device and conveyed through the vascular system as a narrow elongated device, as is shown in FIG. 1b. After the tension is relaxed in situ, upon, for example, release from the catheter, the device at least substantially reverts to its original shape and diameter.

Self-expansible stents according to the present invention may include stents having a braided flexible tubular body. Such stents are further illustrated and described in, for example, U.S. Pat. Nos. 4,655,771, 4,954,126, 4,733,665 and 5,061,275, which are incorporated by reference as if fully set forth herein.

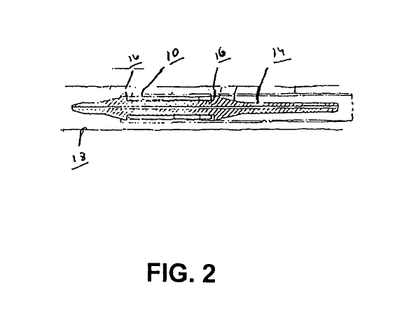

FIG. 2 illustrates an exemplary catheter device 14, which includes stent 10 mounted thereupon. Stent 10, in its elongated configuration (as is shown in FIG. 1b), is mounted in catheter 14. Catheter 14 includes mechanism 16 for mounting and retaining self-expansible stent 10 therein. Catheter 14 is utilized to deliver and position stent 10 within a lumen of, for example, an artery, vein, or any other blood vessel of the human vascular system or any other tissue within the lumen of which catheters and stents can be delivered. Fluoroscopy, and/or other conventional techniques may be utilized to insure that catheter 14 and stent 10, are delivered to the desired location within the lumen. Stent 10 is thereafter released from catheter 14 and self-expands, as described herein, so as to anchor within lumen 18. Thereafter, catheter 14 may be removed from lumen 18.

Forcedly expansible stents are typically flexible tubular bodies that expand upon and forced against a lumen wall by an expandable (e.g., inflatable) portion of a catheter, e.g., a balloon, which fixes it into position. Hence, stent devices having a forcedly expansible stent body are typically mounted on a catheter that includes an inflatable portion, preferably, an angioplasty balloon, and expand upon inflating the balloon.

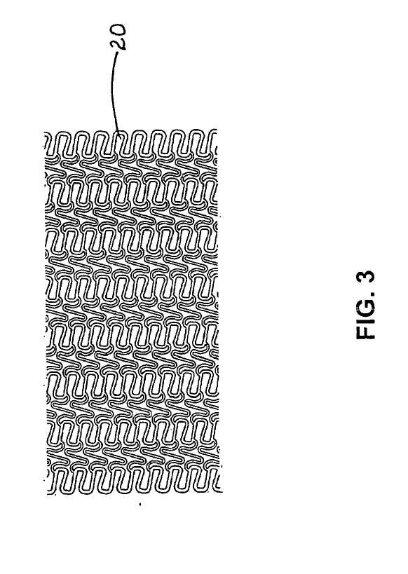

FIG. 3 shows a schematic cross-sectional view of an exemplary forcedly expansible stent 20. Forcedly extensible stents are further described in, for example, U.S. Pat. Nos. 4,733,665, 4,800,882 and 4,886,062.

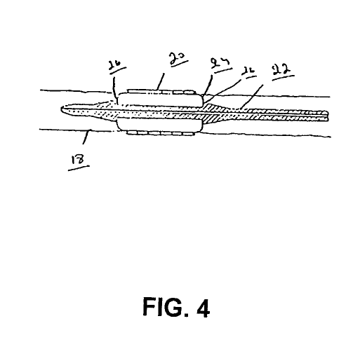

FIG. 4 shows a cross-sectional view of an exemplary catheter device 22, having a forcedly expansible stent 20 mounted thereon. Forcedly expansible stent device 20, is mounted on catheter 22. Catheter 22 includes a conventional angioplasty balloon 24 or any other inflatable device, and a mechanism 26 for mounting and retaining stent 20 over balloon 24. Catheter 22 and stent 20 are delivered to the desired location within lumen 18. Stent 20 is then expanded by expanding the inflatable balloon 24 of catheter 22, whereby stent 22 is forced radially and outwardly into contact with lumen 18. After the desired expansion of stent 22 has been accomplished, angioplasty balloon 24 may be collapsed, or deflated, and catheter 22 may be removed in a conventional manner from lumen 18.

As the above description is meant to illustrate some embodiments of the stent device and the catheter device of the present invention, other configurations of stents and catheters are usable in the context of the present invention.

For example, bifurcation stents and delivery systems designed to position such stents at or close to arterial bifurcations, as for example described in U.S. Pat. Nos. 6,325,826; and 6,210,429; U.S. patent application Ser. Nos. 09/668,832; 09/533,616; 09/455,299; 09/816,690; 09/860,744; 60/329,713; 09/741,761; 60/375,075; 60/339,802; 09/007,265; 09/750,372; 09/600,348; 09/669,060; 09/668,687; 09/827,637; 09/963,114; 09/325,996; 09/663,111; 09/794,740; 10/050,524; 60/208,399; 60/088,301; 60/155,611; 60/208,393; 08/935,383; 08/44,002; and 09/614,472; and PCT Application Nos. US01/09638; IL02/00150; IL02/00223; IL02/00381; US00/15437; US99/00835; US97/18201; US00/26339; US00/26378; and US00/26382, are usable in the context of the present invention. Hence, the description of stent and catheter designs described herein is not to be regarded as limiting.

As mentioned herein, stent device(s) according to the present invention include one or more HDAC inhibitor depot(s) provided in or on the stent body, and, as such, present a novel stent configuration for the prevention and/or treatment of restenosis.

As used herein, the phrase “HDAC inhibitor depot” describes a store of at least one HDAC inhibitor designed to retain and thereafter release the HDAC inhibitor(s).

HDAC inhibitor depot(s) of stent devices of the present invention may be capable of controllably releasing the HDAC inhibitor(s) and the additional pharmaceutical agent(s), if present. Hence, the depots of the present invention can be made of any material that can entrap, encapsulate, adhere or otherwise retain and controllably release the HDAC inhibitor(s).

Preferably, depots of the present invention comprise one or more biocompatible polymer(s) loaded with HDAC inhibitor(s) and/or additional pharmaceutical agent(s). Preferably the biocompatible polymer utilized minimizes irritation to the wall of the lumen where the stent is implanted.

Several loading configurations are envisaged by the present invention.

The HDAC inhibitor(s) can be, for example, molded into the polymer, entrapped or encapsulated within the polymer, covalently attached to the polymer, physically adhered to the polymer or otherwise incorporated into the biocompatible polymer.

The biocompatible polymer can be, for example, either a biostable polymer or a biodegradable polymer, depending on factors such as the desired rate of release or the desired degree of polymer stability under physiological conditions.

Biodegradable polymers that are usable in the context of the present invention include, without limitation, poly(L-lactic add), polycaprolactone, poly(lactide-co-glycolide), poly(hydroxybutyrate), poly(hydroxybutyrate- co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and/or biomolecules such as fibrin, fibrinogen, cellulose, starch, collagen and hyaluronic acid.

Biostable polymers that are usable in the context of the present invention include, without limitation, polyurethanes, silicones, polyesters, polyolefins, polyisobutylene, ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

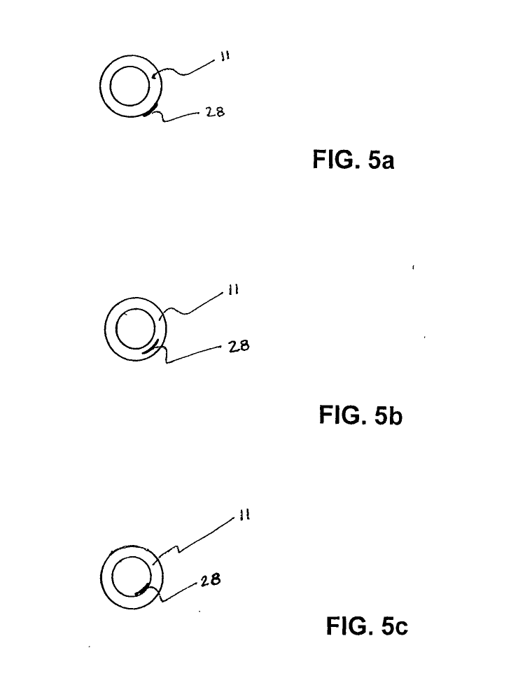

As is described herein and is further schematically illustrated in FIGS. 5a-b, the depot is present either in (FIG. 5b) or on (FIG. 5a) the stent body. FIGS. 5a, 5b, and 5c show a cross section of stent body 11 of stent 10. In FIGS. 5a and 5c, a depot 28 is present on stent body 11, while in FIG. 5b, depot 28 is present within stent body 11. Although FIGS. 5a-c show the depots 28 being present only in or on a portion of the circumference of the stent bodies 11, the depot may extend around the entire, or only a portion of, the stent body circumference. Likewise, the depot may extent for all or only a portion of the length of the stent body. The size of the depot depends on various parameters, such as the material of which the stent body is fabricated, the permeability of the stent body and the depot, the efficacy of the depot in retaining the HDAC inhibitor(s), the concentration of the HDAC inhibitor(s), and the desired rate and duration of release of the HDAC inhibitor(s).

The position of the depot with respect to the stent body also depends on various parameters, such as the material of which the stent body is fabricated, its permeability, the efficacy of the depot in retaining the HDAC inhibitor(s), and the desired rate and duration of release of the HDAC inhibitor(s).

In certain embodiments, the depot is formed from an external surface of the stent body, as is shown, for example, in FIGS. 5a and 5c. In such cases, the HDAC inhibitor depot(s) and/or additional pharmaceutical agent depot(s) can be formed by a coating of the stent device.

One or more pharmaceutical agent depot(s), if present, may likewise be positioned and sized according to the desired results, taking into account parameters such as those taken into account in determining the size and position of the HDAC inhibitor depot(s).

The HDAC inhibitor(s) and/or additional pharmaceutical agent(s) is entrapped, encapsulated, adhered or otherwise retained in the depot(s), in a manner suitable for controllable release of the HDAC inhibitor and/or additional pharmaceutical agent(s) therefrom. The HDAC inhibitor(s) and/or additional pharmaceutical agent(s) can, for example, be molded into a biocompatible polymer, which is thereafter attached to a stent body, to thereby produce a stent device according to the present invention.

As is described herein and is further demonstrated in the Examples section that follows, HDAC inhibitor(s), such as TSA, were found highly effective in inhibiting the proliferation of SMCs. However, it should be noted that any HDAC inhibitor that demonstrates inhibitory effect on cell growth can be used in the context of the depot of the present invention and are therefore encompassed by the present invention.

The presently known HDAC inhibitors are typically classified by their chemical structure. Presently, there are four main classes of HDAC inhibitors: hydroxamic acids and derivatives thereof, short-chain fatty acids and derivatives thereof, cyclic tetrapeptides, and benzamide derivatives. The cyclic tetrapeptide class include two subclasses: cyclic tetrapeptides containing a 2-amino-8-oxo-9,10-epoxy-decanoyl (AOE) moiety and cyclic tetrapeptides not containing the AOE moiety.

Additional HDAC inhibitors that can be used with the present invention include, without limitation, diallyl sulfide and related molecules, valproic acid derivatives, carbamic acid derivatives, derivatives of oxyalkylene esters and oxyalkylene phosphates and structurally unrelated compounds such as depudecin, and psammaplin A.

These HDAC inhibitors are described in detail in, for example, Lea and Tulsyan, 1995, Nokajima et al., 1998, Saito et al., 1999, Suzuki et al., 1999, Lea et al., 1999, WO 00/21979, EP 1170008, WO 02/30879, WO 02/26696, WO 02/26703 and in U.S. Pat. Nos. 6,043,389, 6,030961 and 6,239,176.

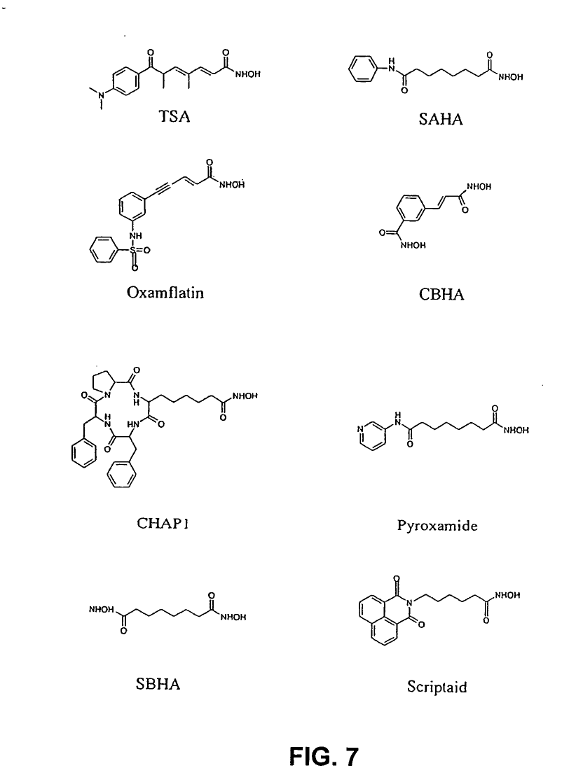

FIG. 7 presents the chemical structures of HDAC inhibitors that are classified as hydroxamic acids, such as trichostatin A (TSA), suberoylanilide hydroxamic acid (SAHA), oxamflatin, m-carboxycinnamic acid bis-hydroxamide (CBHA), cyclic hydroxamic-acid-containing peptide 1 (CHAP1), CHAP31, pyroxamide, suberic bishydroxamate (SBHA), and scriptaid.

The newly-developed compounds known as NVP-LAQ824, CG1521, CG1255, CG1552, and PXD101 which belong to the hydroxamic acids class can also be utilized with the present invention. Other compounds that act as HDAC inhibitors and include functional groups that are derivatives of hydroxamic acid can also serve as a HDAC inhibitor according to the present invention.

In addition, assays described in the Examples section that follows can be used to screen among known and future HDAC inhibitors for efficacy in arresting SMC growth and, thus, suitability for use with the present invention.

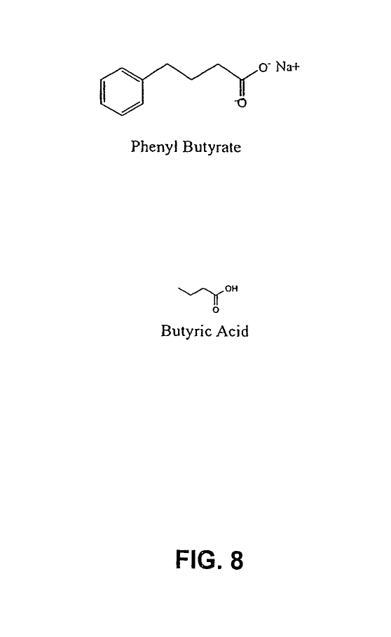

FIG. 8 presents the chemical structures of butyric acid and phenyl butyrate, which are exemplary HDAC inhibitors that are classified as short-chain fatty acids.

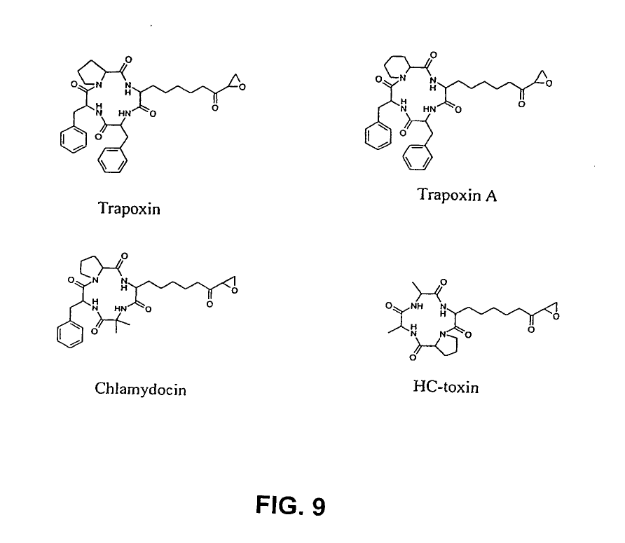

FIG. 9 presents the chemical structures of trapoxin, trapoxin A, chlamydocin and HC toxin, which are representative examples of HDAC inhibitors classified as cyclic tetrapeptides that contain a 2-amino-8-oxo-9,10-epoxy-decanoyl (AOE) moiety.

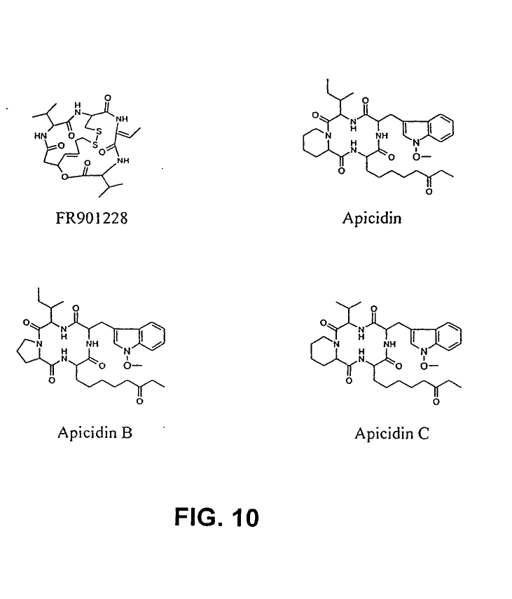

FIG. 10 presents the chemical structures of FR901228, apicidin, apicidin B and apicidin C, which are representative examples of HDAC inhibitors classified as cyclic tetrapeptides not containing a 2-amino-8-oxo-9,10-epoxy-decanoyl (AOE) moiety.

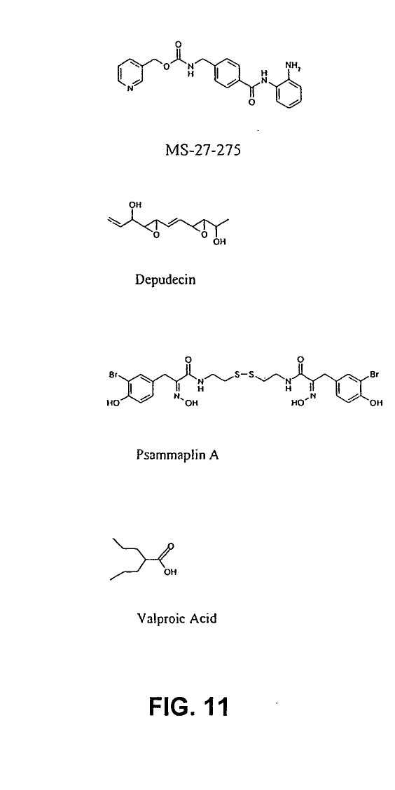

FIG. 11 presents the chemical structure of MS-27-275, which is a representative example of a benzamide derivative HDAC inhibitor, and the chemical structures of depudecin, psammaplin A and valproic acid, described herein.

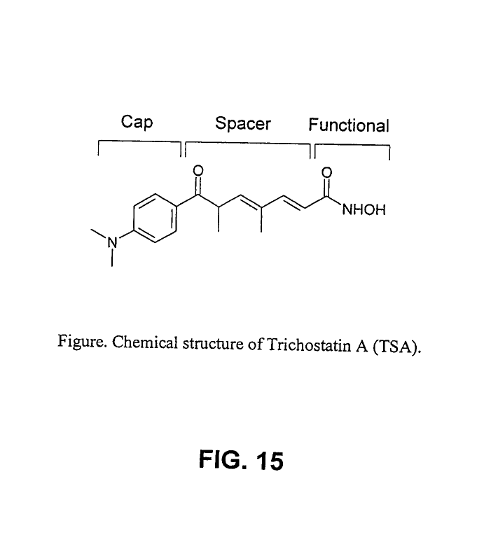

An exemplary HDAC inhibitor preferably that may be used in accordance with the present invention is TSA. FIG. 15 presents the chemical structure of Trichostatin A (TSA). As shown in the Examples section which follows, TSA is highly active in inhibiting human SMC growth and is therefore highly effective in preventing restenosis and in treating restenosis.

Any of the above described HDAC inhibitors can be used alone or in combination with other HDAC inhibitor(s) and/or in combination with other pharmaceutical agents, as described herein.

The HDAC inhibitor depot(s) of the present invention can also include a combination of different HDAC inhibitors or a combination of an HDAC inhibitor and one or more additional pharmaceutical agent(s). Particularly suitable additional pharmaceutical agents include those that are effective in preventing and/or treating restenosis and those that facilitate the treatment and/or prevention of restenosis by the HDAC inhibitor(s). Suitable additional pharmaceutical agents include, without limitation, anticoagulant agents, antiproliferative agents, antimigratory agents, antimetabolic agents, anti-inflammatory agents, and immunosuppressive agents. Exemplary pharmaceutical agents include, for example, rapamycin and paclitaxol.

Such combinations of different HDAC inhibitors or of HDAC inhibitor(s) and other pharmaceutical agent(s) within the HDAC inhibitor depot(s) of the present invention provide for enhanced activity of the stent device in preventing and/or treating restenosis.

Alternatively, the stent device of the present invention can comprise, in addition to the HDAC inhibitor depot(s) described herein, one or more additional pharmaceutical agent depot(s). Such combinations likewise provide for enhanced activity of the stent device in preventing and/or treating restenosis.

Additional pharmaceutical agent depot(s) of the present invention contain one or more pharmaceutical agent(s) and are capable of retaining and controllably releasing these pharmaceutical agent(s). The additional pharmaceutical agent depot(s) can be positioned in or on the stent body, as is further described herein. Pharmaceutical agents suitable for inclusion in these depots include any pharmaceutical agent that is effective in the treatment and/or prevention of restenosis, and any pharmaceutical agent that facilitates the treatment and/or prevention of restenosis by the HDAC inhibitor(s). Exemplary pharmaceutical agents include the pharmaceutical agents described herein, such as, without limitation, anticoagulant agents, antiproliferative agents, antimigratory agents, antimetabolic agents, anti-inflammatory agents, and immunosuppressive agents.

A stent device including a combination of depots provides for enhanced and controllable activity of the stent device by enabling the release of the different active ingredients at different stages, rates and duration.

As is mentioned herein, the stent device of the present invention can be utilized in preventing the occurrence of restenosis in a subject and in treating a subject suffering from restenosis.

Thus, according to another aspect of the present invention, there is provided a method of preventing and/or treating restenosis in a vessel lumen of a subject in need thereof. The method is effected by positioning in the lumen, at or near the site of existing, suspected, or anticipated restenosis, a stent device of the present described herein. Such positioning can be effected using a delivery catheter, such as, but not limited to, the delivery catheters described herein with respect to FIGS. 2 and 4.

The type of stent device and depot configuration utilized and the position of stent anchoring depend upon the type and severity of restenosis and the vessel in which restenosis is observed, suspected, or anticipated.

An ordinarily skilled artisan would be well capable of selecting the appropriate stent and depot configuration suitable for preventing and/or treating specific types of restenosis and as such, no further description of stent/depot configurations suitable for treating such specific types of restenosis need be provided herein in order to enable one of skill in the art to make and practice the invention to its full scope.

Additional objects, advantages, and novel features of the present invention will become apparent to one ordinarily skilled in the art upon examination of the following examples, which are not intended to be limiting. Additionally, each of the various embodiments and aspects of the present invention as delineated and as claimed herein finds experimental support in the following examples.

### EXAMPLES

Reference is now made to the following examples, which together with the above descriptions, illustrate the invention in a non-limiting fashion.

### Materials and Experimental Methods

Cells:

Human aortic SMCs were obtained from BioWhittaker Molecular Applications, Inc. (Clonetics, CC-2571), cultured in Smooth Muscle Medium-2 (BioWhittaker Molecular Applications, Inc., CC-3182), and maintained at 37° C. in humidified 95% air/5% CO₂. Human aortic SMCs of passages 8-9 were used in the following experiments.

Drugs:

TSA (Sigma, T8552), 0.5 mg/ml in 200-proof ethanol. Filtered aliquots were stored at −20° C.

Rapamycin (Calbiochem, 553210, RPM), 10 mM in 200-proof ethanol. Filtered aliquots were stored at −20° C.

Paclitaxel (Sigma, T7402), 100 mM in 200-proof ethanol. Filtered liquots were stored at −20° C.

Measurement:

Cell proliferation was measured by MTT assay and cell counting.

MTT Assay and Morphological Analysis:

Human aortic SMCs cells were seeded, at a density of 6×10³ cells/well, in a 24-well plate (about 3×10³ cells/cm², quadruplicates). Following cell attachment for 24 hours, the above drugs were added to the cells, to achieve the following final concentrations: 100 nM and 500 nM of TSA, 10 nM of RPM, and 50 nM of Paclitaxel.

Following a one day incubation with the drug, the morphological changes in the cells were evaluated under a microscope.

Following three additional days of incubation, 50 μl MTT (methylthiazoletetrazolium, 5 mg/ml in sterile H₂O) were added in 0.5 ml medium to each well, and the cells were incubated at 37° C. for 4 hours. The medium was then aspirated, and the cells were dissolved in 300 μl 0.1 N HCl/isopropanol, mixed thoroughly, and a 100 μl portion of the mixture was transferred into a 96-well plate;

The 96-well plate was applied to a micro-plate reader and the absorbance at 570 nm was read. The average reading of wells containing only medium was set as the Control. For each sample, the proliferation rate was determined by the following equation:
(Reading/Control)×100=Proliferation Rate (% of Control).

Cell Counting:

Human aortic SMCs cells were seeded, at a density of 1.14×10⁴ cells/well, in a 24-well plate (about 1.5×10³ cells/cm², in triplicates). Following cell attachment for 24 hours, TSA was added to achieve a final concentration of 50 nM.

Every 2-3 days, the medium was removed and replaced with a fresh medium containing TSA. Control cells were treated with medium without TSA.

On days 0, 1, 2, 3, 6, 9, and 14 post-treatment, the cells were trypsinized and counted with a hemacytometer.

### Experimental Results

MTT Assay and Morphological Analysis:

The effects of TSA (at different concentrations), RPM and Paclitaxel on the morphology and the proliferation of SMCs were determined by a morphological analysis and by the MTT assay, respectively.

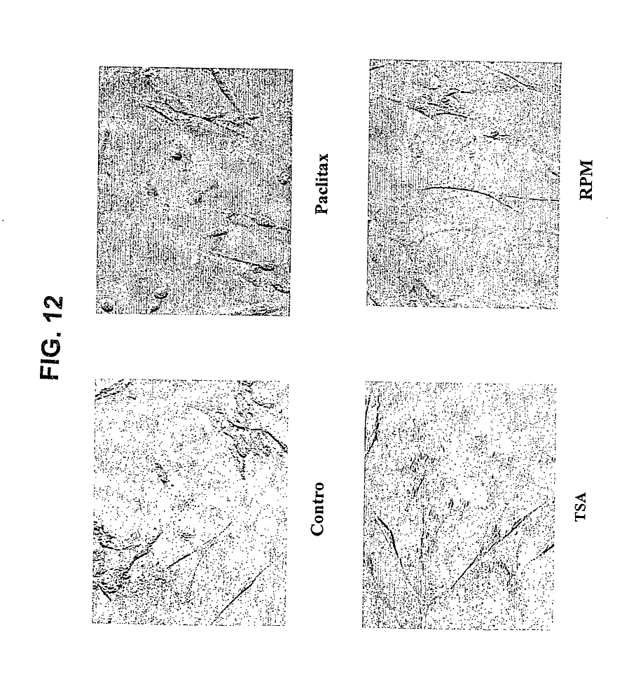

FIG. 12 presents the morphological changes observed in a 40× microscope field following treatment with Paclitaxel, RPM, TSA and no treatment. No morphological changes were observed in cells treated with RPM or TSA and in untreated cells. Rounded up pro-apoptotic cells appeared in the Paclitaxel-treated cells. This morphological analysis demonstrates that the inhibitory effect of RPM and TSA on SMCs is mainly cytostatic.

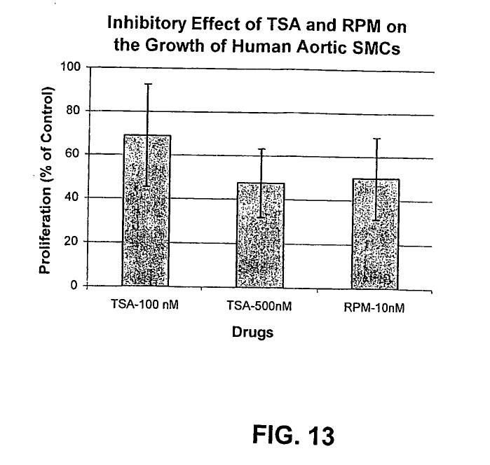

FIG. 13 demonstrates the inhibitory effect of 100 nM TSA, 500 nM TSA and 10 nM RPM on the growth of the human SMCs. The tested dose of Paclitaxel, 50 nM exhibited high cytotoxicity on SMCs and caused cell death before the application of MTT assay. The obtained results show that TSA inhibits SMC proliferation in vitro in a dose-dependent mainer.

Cell Counting:

The continuous effect of 50nM TSA on SMCs growth was further determined by cell counting.

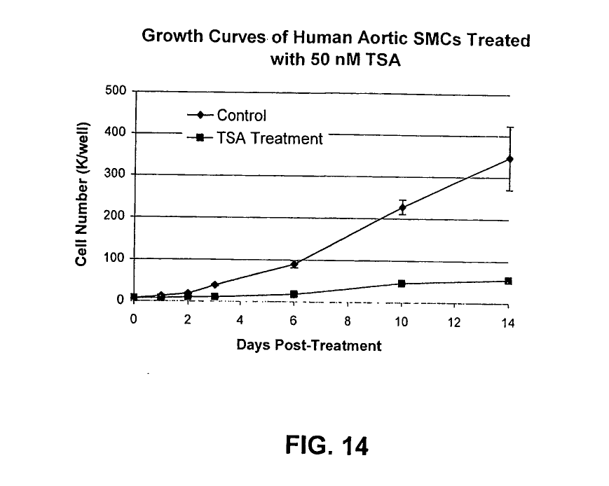

FIG. 14 compares the growth curves obtained for cells treated with TSA and cells treated with medium only (control cells). The growth curves clearly illustrate the significant inhibitory effect of TSA on SMCs at 50nM.

Although the invention has been described in conjunction with specific embodiments thereof, it will be evident to one of skill in the art upon reading the present inventive disclosure that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### References Cited in Alphabetic Order

1. Ailenberg, M. et al., 2002, “Trichostatin A-histone deacetylase inhibitor with clinical therapeutic potential-is also a selective and potent inhibitor of gelatinase A expression”, Biochem Biophys Res Commun., Vol. 298, pp. 110-115;
2. Csordas, A., 1990, “On the biological role of histone acetylation”, Biochem. J., Vol. 265, pp. 23-38;
3. Davie, J. R., 1998, “Covalent modifications of histones: expression from chromatic templates”, Curr. Opin. Genes. Dev., Vol. 8, pp. 173-178;
4. Desai, D. et al., 1999, “Chemopreventive efficacy of suberanilohydroxamic acid (SAHA), a cytodifferentiating agent, against tobacco-specific nitrosamine 4-(methylnitros-amino)-1-(3-pyridyl)-1-butanone (NNK)-induced lung tumorigenesis.” Proc. AACR, Vol. 40, abstract No. 2396;
5. Dichek, D. A. et al., 1989, “Seeding of Intravascular Stents With Genetically Engineered Endothelial Cells”, Circulation, Vol. 80, pp. 1347-1353;
6. Finnin, M. S. et al., 1999, “Structures of a histone deacetylase inhibitor homologue bound to the TSA and SAHA inhibitors”, Nature, Vol. 401, pp. 188-193;
7. Grozinger, C. M. et al., 1999, “Three proteins define a class of human histone deacetylases related to yeast Hda1p”, Proc. Natl. Acad. Sci. USA, Vol. 96, pp. 4868-4873;
8. Kao, H. Y. et al., 2000, “Isolation of a novel histone deacetylase reveals that class I and class II deacetylases promote SMRT-mediated repression”, Genes and Dev., Vol. 14, pp. 55-66;
9. Kelly, W. K. et al., 2001, “Suberoylanilide Hydroxamic Acid (SAHA), a Histone Deacetylase Inhibitor: Biologic Activity Without Toxicity” American Society of Clinical Oncology (2001 Abstract database), Abstract No. 344-2001;
10. Jung, M., 2001, “Inhibitors of histone deacetylase as new anticancer agents”, Curr. Med Chem., Vol. 8, pp. 1505-1511;
11. Kim, Y. B. et al., 1999, “Oxamflatin is a novel antitumor compound that inhibits mammalian histone deacetylase”, Oncogene, Vol. 18, pp. 2461-2470;
12. Kimura, M. et al., 1994, “Dual modes of action of platelet-derived growth factor and its inhibition by trichostatin-A for DNA synthesis in primary cultured smooth muscle cells of rat aorta”, Biol. Pharm. Bull., Vol. 17, pp. 399-402;
13. Kouzarides, T., 1999, “Histone acetylases and deacetylases in cell proliferation”, Curr. Opin. Genet. Dev., Vol. 9, pp. 40-48;
14. Koyama, Y. et al., 2000, “Histone deacetylase inhibitors suppress IL-2-mediated gene expression prior tom introduction of apoptosis”, Blood, Vol. 96, pp. 1490-1495;
15. Lea, M. A. and Tulsyan, N., 1995, “Discordant effects of butyrate analogues on erythroleukemia cell proliferation, differentiation and histone acetylase”, Anticancer Res., Vol. 15, pp. 879-883;
16. Lea, M. A. et al., 1999, “Increased acetylation of histones induces by diallyl disulfide and structurally related molecules”, Int. J. Oncol., Vol. 2, pp. 347-352;
17. Liu, L. T. et al., 2003, “Histone deacetylase inhibitor up-regulates RECK to inhibit MMP-2 activation and cancer cell invasion”, Cancer Res., Vol. 63, pp. 3069-3072;
18. Marks, P. A. et al., 2001a, “Histone deacetylases and cancer: causes and therapies”, Nat. Reviews, Vol. 1, pp. 194-202;
19. Marks, P. A. et al., 2001b, “Histone deacetylase inhibitors as new cancer drugs”, Curr. Opin. Oncol., Vol. 13, pp. 477-483;
20. Marks, P. A. et al., 2000, “Histone deacetylase inhibitors: inducers of differentiation or apoptosis of transformed cells”, J. Natl. Cancer Inst., Vol. 92, pp. 1210-1216;
21. Nakajima, H. et al., 1998, “FR901228, a potent antitumor antibiotic, is a novel histone deacetylase inhibitor”, Exp. Cell Res., Vol. 241, pp. 126-133;
22. Okabe, M. et al., 1995, “Competence effect of PDGF on Ki-67 antigen and DNA contents, and its inhibition by trichostatin-A and a butylydene phthalide BP-421 in primary smooth muscle cells of rat aorta by flow cytometry”, Biol. Pharm. Bull., Vol. 18, pp. 1665-1670;
23. Pazin, M. J., et al., 1997, “What's up and down with histone deacetylation and transcription?“, Cell, Vol. 89, pp. 325-328;
24. Richon, V. M. et al., 1996, “Second generation hybrid polar compounds are potent inducers of transformed cell differentiation”, Proc. Natl. Acad. Sci. USA, Vol. 93, pp. 5705-5708;
25. Richon, V. M. et al., 1998, “A class of hybrid polar inducers of transformed cell differentiation inhibits histone deacetylases”, Proc. Natl. Acad. Sci. USA, Vol. 95, pp. 3003-3007;
26. Saito, A. et al., 1999, “A synthetic inhibitor of histone deacetylase, MS-27-275, with marked in vivo antitumor activity against human tumors”, Proc. Natl. Acad. Sci. USA, Vol. 96, pp. 4592-4597;
27. Skaletz-Rosowski, A. et al., 2000, “The histone deacetylase inhibitors, trichostatin A and the new synthetic inhibitor M232, suppress the proliferation of coronary smooth muscle cells”, European Heart Journal, Vol. 21, Abstract No. P1551;
28. Spencer, V. A. and Davie, J. R., 1999, “Role of covalent modifications of histones in regulating gene expression”, Gene, Vol. 240, pp. 1-12;
29. Suzuki, T. et al., 1999, “Synthesis and histone deacetylase inhibitory activity of new benzamide derivatives”, J. Med. Chem., Vol. 42, pp. 3001-3003;
30. Taunton, J. et al., 1996, “A mammalian histone deacetylase related to the yeast transcriptional regulator Rpd3p”, Science, Vol. 272, pp. 408-411;
31. Takahashi I. et al., 1996, “Selective inhibition of IL-2 gene expression by trichostatin A, a potent inhibitor of mammalian histone deacetylase”, J. Antibiot., Vol. 49, pp. 453-457;
32. Van den Wyngaert, I. et al., 2000, “Cloning and characterization of human histone deacetylase 8”, FEBS, Vol. 478, pp. 77-83;
33. Vigushin, D. M. et al., 2001, “Trichostatin A is a histone deacetylase inhibitor with potent anti-tumor activity against breast cancer in vivo”, Clinical Cancer Research, Vol. 7, pp. 971-976;
34. Wang, C. et al., 2001, “Histone acetylation and the cell-cycle in cancer”, Front Biosci., Vol. 6, pp. D610-29;
35. Warrell, R. P. Jr. et al., 1998, “Therapeutic targeting of transcription in acute promyelocytic leukemia by use of an inhibitor of histone deacetylase”, J. Natl. Cancer Inst., Vol. 90, pp. 1621-1625;
36. Weidle, U. H. et al., 2000, “Inhibition of histone deacetylases: a new strategy to target epigenetic modifications for anticancer treatment”, Anticancer Res., Vol. 20, pp. 1471-1485;
37. Yoshida, M. and Beppu, T., 1988, “Reversible arrest of proliferation of rat 3Y1 fibroblasts in both G1 and G2 phases by trichostatin A”, Exp. Cell. Res. Vol. 177, pp. 122-131;
38. Yoshida, M. et al., 1990, “Potent and specific inhibition of mammalian histone deacetylase both in vivo and in vitro by trichostatin A”, J. Biol. Chem., Vol. 265, pp. 17174-17179;
39. Yoshida, M. et al., 1995, “Trichostatin A and trapoxin: novel chemical probes for the role of histone acetylation in chromatin structure and function”, Bioassays, Vol. 17, pp. 423-430;
40. Yoshida, M. et al., 2001, “Histone deacetylase as a new target for cancer chemotherapy”, Cancer Chemother. Pharmacol., Vol. 48 Suppl 1, pp. S20-S26.

1. A stent device comprising a stent body and one or more HDAC inhibitor depots, said one or more HDAC inhibitor depots being located on or in said stent body, said one or more HDAC inhibitor depots comprising one or more HDAC inhibitors, and said one or more HDAC inhibitor depots being capable of controllably releasing at least one of said one or more HDAC inhibitors.

2. The stent device of claim 1, wherein at least one of said one or more HDAC inhibitor depots further comprises one or more additional pharmaceutical agents, at least one of said one or more additional pharmaceutical agents selected from the group consisting of an anticoagulant agent, an antiproliferative agent, an antimigratory agent, an antimetabolic agent, an anti-inflammatory agent and an immunosuppressive agent.

3. The stent device of claim 2, wherein at least one of said one or more HDAC inhibitor depots is capable of controllably releasing at least one of said one or more additional pharmaceutical agents.

4. The stent device of claim 1, further comprising one or more additional pharmaceutical agent depots, said one or more additional pharmaceutical agent depots being located on or in said stent body, said one or more additional pharmaceutical agent depots comprising one or more additional pharmaceutical agents, and at least one of said one or more additional pharmaceutical agent depots being capable of controllably releasing at least one of said one or more additional pharmaceutical agents.

5. The stent device of claim 4, wherein at least one of said one or more additional pharmaceutical agents is selected from the group consisting of an anticoagulant agent, an antiproliferative agent, an antimigratory agent, an antimetabolic agent, an anti-inflammatory agent and an immunosuppressive agent.

6. The stent device of claim 1, wherein at least one of said one or more HDAC inhibitor depots comprises one or more biocompatible polymers loaded with at least one of said one or more HDAC inhibitors.

7. (Cancelled)

8. (Cancelled)

9. (Cancelled)

10. (Cancelled)

11. (Cancelled)

12. The stent device of claim 1, wherein at least one of said one or more HDAC inhibitors is a hydroxamic acid.

13. The stent device of claim 12, wherein said hydroxamic acid is selected from the group consisting of TSA, SAHA, oxamflatin, CBHA, CHAP1, CHAP31, SBHA, pyroxamide, and scriptaid.

14. The stent device of claim 13, wherein said hydroxamic acid is TSA.

15. The stent device of claim 1, wherein at least one of said one or more HDAC inhibitors is selected from the group consisting of a short-chain fatty acid, a cyclic tetrapeptide, a benzamide derivative, a valproic acid derivative, a carbamic acid derivative, an allyl sulfide-containing compound, an oxyalkylene ester derivative, and an oxyalkylene phosphate derivative.

16. (Cancelled)

17. (Cancelled)

18. (Cancelled)

19. The stent device of claim 1, wherein said stent body comprises a polymer.

20. (Cancelled)

21. (Cancelled)

22. (Cancelled)

23. A stent delivery system comprising:
(a) a delivery catheter; and
(b) a stent device comprising a stent body and one or more HDAC inhibitor depots, said one or more HDAC inhibitor depots being located on or in said stent body, said one or more HDAC inhibitor depots comprising one or more HDAC inhibitors, and said one or more HDAC inhibitor depots being capable of controllably releasing at least one of said one or more HDAC inhibitors,
wherein said stent device is capable of being detachably attached to said delivery catheter.

24. The stent delivery system of claim 23, wherein at least one of said one or more HDAC inhibitor depots further comprises one or more additional pharmaceutical agents, at least one of said one or more additional pharmaceutical agents selected from the group consisting of an anticoagulant agent, an antiproliferative agent, an antimigratory agent, an antimetabolic agent, an anti-inflammatory agent and an immunosuppressive agent.

25. The stent delivery system of claim 24, wherein at least one of said one or more HDAC inhibitor depots is capable of controllably releasing at least one of said one or more additional pharmaceutical agents.

26. The stent delivery system of claim 23, wherein said stent device further comprises one or more additional pharmaceutical agent depots, said one or more additional pharmaceutical agent depots being located on or in said stent body, said one or more additional pharmaceutical agent depots comprising one or more additional pharmaceutical agents, and at least one of said one or more additional pharmaceutical agent depots being capable of controllably releasing at least one of said one or more additional pharmaceutical agents.

27. The stent delivery system of claim 26, wherein at least one of said one or more additional pharmaceutical agents is selected from the group consisting of an anticoagulant agent, an antiproliferative agent, an antimigratory agent, an antimetabolic agent, an anti-inflammatory agent and an immunosuppressive agent.

28. The stent delivery system of claim 23, wherein at least one of said one or more HDAC inhibitor depots comprises one or more biocompatible polymers loaded with at least one of said one or more HDAC inhibitors.

29. The stent delivery system of claim 28, wherein at least one of said one or more biocompatible polymers is a biostable polymer.

30. The stent delivery system of claim 28, wherein at least one of said one or more biocompatible polymers is a biodegradable polymer.

31. The stent delivery system of claim 26, wherein at least one of said one or more additional pharmaceutical agent depots comprises one or more biocompatible polymers loaded with at least one of said one or more additional pharmaceutical agents.

32. (Cancelled)

33. (Cancelled)

34. (Cancelled)

35. (Cancelled)

36. (Cancelled)

37. (Cancelled)

38. (Cancelled)

39. (Cancelled)

40. (Cancelled)

41. (Cancelled)

42. (Cancelled)

43. (Cancelled)

44. (Cancelled)

45. (Cancelled)

46. (Cancelled)

47. (Cancelled)

48. (Cancelled)

49. (Cancelled)

50. (Cancelled)

51. (Cancelled)

52. (Cancelled)

53. (Cancelled)

54. (Cancelled)

55. (Cancelled)

56. (Cancelled)

57. (Cancelled)

58. (Cancelled)

59. (Cancelled)

60. (Cancelled)

61. (Cancelled)

62. (Cancelled)

63. (Cancelled)

64. (Cancelled)

65. (Cancelled)

66. (Cancelled)

67. (Cancelled)
